SEQUENCE LISTING (7 sequences)

SEQ ID NO: 1 - DNA, 65 bp, Artificial Sequence
  NM02 aptamer
tagggaagagaaggacatatgattgattgtccctgttatacgtttgactagtacatgaccacttg

SEQ ID NO: 2 - DNA, 65 bp, Artificial Sequence
  NM01 aptamer
tagggaagagaaggacatatgatgcggttacctgtgtgaccattgactagtacatgaccacttga

SEQ ID NO: 3 - DNA, 65 bp, Artificial Sequence
  NM03 aptamer
tagggaagagaaggacatatgatgcatctactcaacccgattttgactagtacatgaccacttga

SEQ ID NO: 4 - DNA, 62 bp, Artificial Sequence
  NM04 aptamer
tagggaagagaaggacatatgatgcggtttacaccgggtttgactagtacatgaccacttga

SEQ ID NO: 5 - DNA, 63 bp, Artificial Sequence
  NM05 aptamer
tagggaagagaaggacatatgatgattgtaactgttcgttttgactagtacatgaccacttga

SEQ ID NO: 6 - DNA, 23 bp, Artificial Sequence
  Forward primer
tagggaagagaaggacatatgat

SEQ ID NO: 7 - DNA, 23 bp, Artificial Sequence
  Reverse Primer
tcaagtggtcatgtactagtcaa

(19) United States
(12) Patent Application Publication
(10) Pub. No.: US 2024/0167042 A1
(43) Pub. Date: May 23, 2024

(54) DEVELOPMENT OF APTAMERS FOR NEUTRALIZING ANTIBODIES IN DEMYELINATING DISEASES

(71) Applicant: ARIZONA BOARD OF REGENTS ON BEHALF OF THE UNIVERSITY OF ARIZONA, Tucson, AZ (US)

(72) Inventors: May Khanna, Tucson, AZ (US); Kathleen Rodgers, Tucson, AZ (US); Niloufar Mollasalehi, Tucson, AZ (US); Maira Soto, Tucson, AZ (US)

(21) Appl. No.: 18/549,335

(22) PCT Filed: Apr. 11, 2022
(86) PCT No.: PCT/US2022/024237

§ 371 Date: Sep. 6, 2023

Related U.S. Application Data
(60) Provisional application No. 63/173,283, filed on Apr. 9, 2021.

(51) Int. Cl. C12N 15/115; A61K 47/64; C12N 15/10

(52) U.S. Cl. CPC C12N 15/115; A61K 47/64; C12N 15/1048; C12N 2310/16; C12N 2310/3513

(57) ABSTRACT

Multiple sclerosis (MS) is an autoimmune, neurodegenerative disease affecting at least 400,000 individuals in the United States and 2.3 million persons worldwide. Its counterpart in the peripheral nervous system, chronic Inflammatory demyelinating polyradiculoneuropathy (CIPD), affects approximately 40,000 patients In the United States. Both diseases have a relatively young age at diagnosis and require lifelong therapy to slow progression. These two diseases both result from the immune system attacking the integrity of the myelin sheath, the protective coating of neurons. The therapies common to these two diseases are corticosteroids, and plasma exchange/IVIG therapy for the treatment of symptoms, and they are generally immunosuppressive. Hence, there is a need to develop a therapy that would selectively inhibit the specific pathological entities like autoantibodies that bind to the myelin sheath. The present invention features aptamer compositions and methods that can neutralize autoantibodies that target proteins on the myelin sheath.

### CROSS-REFERENCES TO RELATED APPLICATIONS

This application claims benefit of U.S. Provisional Application No. 63/173,283 filed Apr. 9, 2021, the specification of which is incorporated herein in its entirety by reference.

### REFERENCE TO A SEQUENCE LISTING

Applicant asserts that the information recorded in the form of an Annex C/ST.25 text file submitted under Rule 13ter.1(a), entitled >>>UNIA_21_04_PCT_Sequence_Listing_ST25<<<, is identical to that forming part of the international application as filed. The content of the sequence listing is incorporated herein by reference in its entirety.

### FIELD OF THE INVENTION

The present invention features compositions and methods for treating demyelinating diseases, specifically, the present invention features aptamers for neutralizing autoantibodies.

### BACKGROUND OF THE INVENTION

Multiple Sclerosis (MS) is an autoimmune demyelinating disease of the central nervous system affecting between 400,000-1 million Americans and ˜2.3 million people worldwide. So far, the most widely used treatments for MS consist of suppression or modulation of the immune system and ameliorating symptoms. Therapy should be initiated early in the course of the disease to prevent continuing demyelination and secondary axonal loss leading to permanent disability. Autoantibodies in MS patients induce conduction block and demyelination in neurons by attacking the myelin sheath components, potentially through blocking epitopes that are functionally relevant for nerve conduction. Among the potential targets of autoantibodies are myelin oligodendrocyte glycoprotein (MOG) 7, neurofascin 8, contactin-2 9, and the potassium channel, KIR-4.1

The therapies for this disease are generally non-specific in nature and broadly affect the immune function or inflammatory processes leading to significant side effects that can be debilitating to the patient. Further, a considerable proportion of patients continue to progress even while receiving these therapies. Therefore, a therapy that would selectively inhibit the specific pathological entities causing disease tailored to the patient would be a revolutionary advance in therapeutic options. The present invention features methods and compositions that target autoantibodies against myelin oligodendrocyte glycoprotein (MOG) directly correlated to disease outcomes which will alter the course of the disease without immunocompromising the patient.

### BRIEF SUMMARY OF THE INVENTION

It is an objective of the present invention to provide compositions and methods that allow for targeted treatments of demyelinating diseases, as specified in the independent claims. Embodiments of the invention are given in the dependent claims. Embodiments of the present invention can be freely combined with each other if they are not mutually exclusive.

MOG is a member of the immunoglobulin (Ig) superfamily, and a myelin protein expressed only at the external surface of myelin sheaths and oligodendrocyte membranes. The function of MOG is not fully understood, but its molecular structure and its extracellular immunoglobulin (Ig) domain suggests a possible function as a cell surface receptor or in cell adhesion. MOG has a large N-terminal extracellular immunoglobulin G variable (IgG V) like domain (aa 1-125) that is responsible for the formation of demyelinating autoantibodies (Abs).

Aptamers are single-stranded oligonucleotides that can adapt to any structural conformation, bind to target molecules, and are often called “nucleic acid antibodies”. The approach of the present invention is to develop aptamers using the systematic evolution of ligands by exponential enrichment (SELEX) as an in vitro selection method to neutralize MOG-specific 8-18 C5 antibodies. The process of SELEX begins with an extensive library of random sequences ranging in length from 15-100 nucleotides. Following multiple rounds of selection with increasing stringency, aptamers against specific targets and high affinity can be discovered. Advantages of aptamers include (i) potential to bind to a wide range of molecules with high affinity, (ii) small size (ten times smaller than antibodies, permitting flux across cells), (iii) high malleability, and (iv) low cost of production. Also, unlike protein therapeutics, aptamers do not evoke an antigenic response, as shown in several recent in vivo studies.

In some embodiments, the present invention features an aptamer that neutralizes autoantibodies in the peripheral nervous system (PNS) and/or the central nervous system (CNS). The present invention may feature an aptamer that neutralizes autoantibodies in the brain. In other embodiments, the present invention features an aptamer that neutralizes myelin oligodendrocyte glycoprotein (MOG) 7 recognizing autoantibodies. In further embodiments, the present invention features an aptamer that neutralizes neurofascin (NFASC) recognizing autoantibodies.

In other embodiments, the present invention features a method of treating a demyelinating disease in a subject in need thereof. The method may comprise administering an effective amount of an aptamer that neutralizes autoantibodies (e.g., MOG7 or NFASC) in the central nervous system (e.g., the brain). In some embodiments, the present invention features a method of treating multiple sclerosis (MS) in a subject in need thereof, the method comprising administering an effective amount of an aptamer that neutralizes myelin oligodendrocyte glycoprotein (MOG) 7 recognizing autoantibodies. In other embodiments, the present invention features a method of treating chronic inflammatory demyelinating polyradiculoneuropathy (CIDP) in a subject in need thereof, the method comprising administering an effective amount of an aptamer that neutralizes Neurofascin (NFASC) recognizing autoantibodies.

In further embodiments, the present invention features a method for identifying an aptamer for neutralizing autoantibodies in the peripheral nervous system (PNS) and/or central nervous system (CNS; e.g., the brain). The present invention may also feature a method for identifying a high-affinity aptamer against myelin oligodendrocyte glycoprotein (MOG) 7 recognizing autoantibodies.

One of the unique and inventive technical features of the present invention is the use of aptamers to target antibodies directly. Without wishing to limit the invention to any theory or mechanism, it is believed that the technical feature of the present invention advantageously provides for targeted treatment of a demyelinating disease. None of the presently known prior references or work has the unique, inventive technical feature of the present invention.

In fact, the prior references teach away from the present invention. For example, current treatments for demyelinating diseases are general immunosuppressives that target the immune system as a whole.

Furthermore, the inventive technical features of the present invention contributed to a surprising result. For example, the present invention features the first example of an aptamer used to target an auto-antibody. Moreover, prior references, in the field, target proteins, such as MOG for which auto-antibodies develop. The present invention, however, develops aptamers to bind auto-antibodies.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIGS. 1A, 1B, and 1C show flow cytometry can measure the improvement of aptamers' affinity towards the antibody over starting library (SL). Flow cytometry assay was used for Alexa-657 labeled aptamers to measure background binding using only flow cytometry antibody capture beads (FIG. 1A), starting library binding to MOG antibody immobilized on the beads (FIG. 1B), and aptamers from round 6 binding to MOG antibody immobilized on the beads (FIG. 1C). The comparison between round 6 and starting library data shows 9 times enrichment of binding of aptamers to the MOG antibody observed as a higher percentage of beads showing high Alexa signal on x-axis due to binding of Alexa-657 labeled aptamers to the antibodies immobilized on the beads.

FIG. 2 shows a radioactive filter binding assay that measures the improvement of aptamer affinity towards the antibody over the starting library (SL). Radioactive filter binding assay was used to confirm enrichment of aptamers with high affinity towards MOG antibody in round 16 (grey) compared to SL (black). The quantification of radioactive filter binding assay in the absence or presence of 10 μM MOG antibody for round 16 showed 2× enrichment in the aptamer pool.

FIGS. 3A, 3B, and 3C show the surface plasmon resonance (SPR) studies of sequenced aptamer and binding to MOG antibody. MOG antibody was immobilized on the surface of a CM5 chip (9000 RU), and IgG Fc fragment was immobilized on the surface of the control flow cell on the chip. FIG. 3A shows SPR sensorgrams showing the signal for binding of different aptamers at 10 μM to immobilized MOG antibodies. The sequence of each aptamer with underlined portions identifying the constant region in each sequence is shown (below; SEQ ID NO: 1-5). FIG. 3B. shows a representative SPR sensorgram showing the signal for binding of different concentrations of the aptamer (1-100 UM) and immobilized MOG antibody. FIG. 3C shows a concentration-dependent curve that was obtained for aptamer binding to immobilized MOG antibodies showing Kd=11±24 μM. At higher concentrations of the aptamer, it started to show non-specific binding signals; thus, the data is not shown. Data are represented as mean±SEM (n=3). Some error bars are smaller than the symbols.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

The terms “administering” and “administration” refer to methods of providing a pharmaceutical preparation to a subject. Such methods are well known to those skilled in the art and include, but are not limited to, administering the compositions orally, parenterally (e.g., intravenously and subcutaneously), by intramuscular injection, by intraperitoneal injection, intrathecally, transdermally, extracorporeally, topically or the like.

As used herein, the terms “treat” or “treatment” or “treating” refers to both therapeutic treatment and prophylactic or preventative measures, wherein the object is to prevent or slow the development of the disease, such as slow down the development of a disorder, or reducing at least one adverse effect or symptom of a condition, disease or disorder, e.g., any disorder characterized by insufficient or undesired organ or tissue function. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease but also a cessation or slowing of progress or worsening of a symptom that would be expected in the absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” also includes ameliorating a disease, lessening the severity of its complications, preventing it from manifesting, preventing it from recurring, merely preventing it from worsening, mitigating an inflammatory response included therein, or a therapeutic effort to affect any of the aforementioned, even if such therapeutic effort is ultimately unsuccessful.

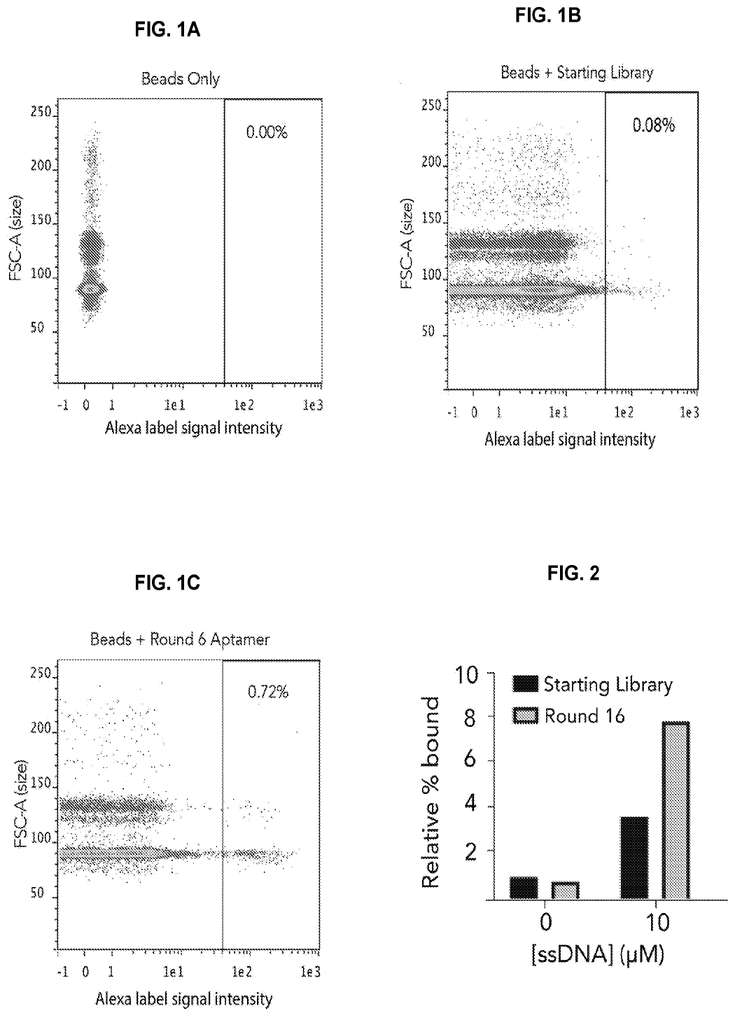

Referring now to FIGS. 1A-3C, the present invention features compositions and methods for targeting and neutralizing autoantibodies using aptamers.

The present invention features an aptamer that neutralizes autoantibodies in the peripheral nervous system (PNS) and/or the central nervous system (CNS). The present invention may feature an aptamer that neutralizes autoantibodies in the brain. In other embodiments, the present invention features an aptamer that neutralizes myelin oligodendrocyte glycoprotein (MOG) 7 recognizing autoantibodies. In further embodiments, the present invention features an aptamer that neutralizes neurofascin (NFASC) recognizing autoantibodies.

Without wishing to limit the present invention to any theory or mechanism it is believed that the use of the aptamers described herein predominantly in the peripheral nervous system would overcome challenges with crossing the blood brain barrier (BBB).

As used herein, “aptamer” may refer to short, single-stranded oligonucleotides that can selectively bind to a specific target, including but not limited to proteins, peptides, carbohydrates, small molecules, or toxins. An aptamer may be DNA or RNA.

TABLE 1

| | | SEQ |
|---|---|---|
| | | ID |
| Aptamer: | Sequence: | NO: |
| NM02 | TAGGGAAGAGAAGGACATATGATTGATTGTCCCTGTT | 1 |
| | ATACGTTTGACTAGTACATGACCACTTG | |
| NM01 | TAGGGAAGAGAAGGACATATGATGCGGTTACCTGTGT | 2 |
| | GACCATTGACTAGTACATGACCACTTGA | |
| NM03 | TAGGGAAGAGAAGGACATATGATGCATCTACTCAACC | 3 |
| | CGATTTTGACTAGTACATGACCACTTGA | |
| NM04 | TAGGGAAGAGAAGGACATATGATGCGGTTTACACCGG | 4 |
| | GTTTGACTAGTACATGACCACTTGA | |
| NM05 | TAGGGAAGAGAAGGACATATGATGATTGTAACTGTTC | 5 |
| | GTTTTGACTAGTACATGACCACTTGA | |

In some embodiments, the aptamer comprises nucleotides. In other embodiments, the aptamer comprises single-stranded deoxyribonucleic acid (ssDNA). In other embodiments, the aptamer comprises single-stranded ribonucleic acid (ssRNA). In other embodiments, the aptamer comprises SEQ ID NO: 1, or SEQ ID NO: 2, or SEQ ID NO: 3, or SEQ ID NO: 4, or SEQ ID NO: 5. In preferred embodiments, the aptamer comprises SEQ ID NO: 1.

In some embodiments, the aptamer is 100% identical to any of the aforementioned sequences (SEQ ID NO: 1-5) or a fragment thereof. In some embodiments, the aptamer is at least 98% identical to any of the aforementioned sequences (SEQ ID NO: 1-5) or a fragment thereof. In some embodiments, the aptamer is at least 95% identical to any of the aforementioned sequences (SEQ ID NO: 1-5) or a fragment thereof. In some embodiments, the aptamer is at least 90% identical to any of the aforementioned sequences (SEQ ID NO: 1-5) or a fragment thereof. In some embodiments, the aptamer is at least 85% identical to any of the aforementioned sequences (SEQ ID NO: 1-5) or a fragment thereof. In some embodiments, the aptamer is at least 80% identical to any of the aforementioned sequences (SEQ ID NO: 1-5) or a fragment thereof. In some embodiments, the aptamer is at least 75% identical to any of the aforementioned sequences (SEQ ID NO: 1-5) or a fragment thereof. In some embodiments, the aptamer is at least 70% identical to any of the aforementioned sequences (SEQ ID NO: 1-5) or a fragment thereof.

In some embodiments, the aptamer is 100% identical to SEQ ID NO: 1 or a fragment thereof. In some embodiments, the aptamer is at least 98% identical to SEQ ID NO: 1 or a fragment thereof. In some embodiments, the aptamer is at least 95% identical to SEQ ID NO: 1 or a fragment thereof. In some embodiments, the aptamer is at least 93% identical to SEQ ID NO: 1 or a fragment thereof. In some embodiments, the aptamer is at least 90% identical to SEQ ID NO: 1 or a fragment thereof. In some embodiments, the aptamer is at least 88% identical to SEQ ID NO: 1 or a fragment thereof. In some embodiments, the aptamer is at least 85% identical to SEQ ID NO: 1 or a fragment thereof. In some embodiments, the aptamer is at least 83% identical to SEQ ID NO: 1 or a fragment thereof. In some embodiments, the aptamer is at least 80% identical to SEQ ID NO: 1 or a fragment thereof. In some embodiments, the aptamer is at least 78% identical to SEQ ID NO: 1 or a fragment thereof. In some embodiments, the aptamer is at least 75% identical to SEQ ID NO: 1 or a fragment thereof. In some embodiments, the aptamer is at least 73% identical to SEQ ID NO: 1 or a fragment thereof. In some embodiments, the aptamer is at least 70% identical to SEQ ID NO: 1 or a fragment thereof.

In some embodiments, the autoantibodies are myelin oligodendrocyte glycoprotein (MOG) 7 recognizing autoantibodies. In further embodiments, autoantibodies are Neurofascin (NFASC) recognizing autoantibodies.

In some embodiments, the aptamer may further comprise a peptide. In other embodiments, the aptamer comprises a peptide moiety. In some embodiments, the peptide is coupled to the aptamer (e.g., the nucleic acid). In some embodiments, the peptide moiety is coupled to the aptamer (e.g., the nucleic acid). In some embodiments, the aptamer comprises a peptide coupled to said aptamer (e.g., the nucleic acid). In some embodiments, the aptamer comprises a peptide moiety coupled to said aptamer (e.g., the nucleic acid). In some embodiments, the aptamer comprises a cell-penetrating peptide (CPP). In other embodiments, a CPP is a short peptide that facilitates cellular intake and uptake of molecules. In some embodiments, the peptide increases the ability of the aptamer to cross the blood-brain barrier (BBB).

Without wishing to limit the present invention to any theory or mechanism it is believed that because the CCP peptide is derived from HIV (human immunodeficiency virus), it makes the aptamer comprising said CCP peptide efficient at cell penetration as well as BBB crossing. The CPP peptide may comprise arginine residues (R8) and allow any cargo (e.g., aptamers described herein) to penetrate the cell via endocytosis.

In some embodiments, the peptide is directly attached to the aptamer. In other embodiments, the peptide is attached to the aptamer via a linker. In further embodiments, the peptide is attached to the aptamer via a short linker. In some embodiments, the linker (e.g., the short linker) is about 5 to 15 amino acids long. In other embodiments, the linker (e.g., the short linker) is about 8 to 12 amino acids long. The linker length may be optimized to ensure maximum uptake of the aptamer described herein.

In some embodiments, the aptamer prevents demyelination. In other embodiments, the aptamer treats demyelinating diseases. In further embodiments, the demyelinating diseases may include but are not limited to multiple sclerosis (MS) or chronic inflammatory demyelinating polyradiculoneuropathy (CIDP). In other embodiments, the aptamers may be used to prevent/treat diabetes e.g., aptamers may be developed for anti-insulin autoantibodies.

In other embodiments, the aptamer may be useful at preventing other diseases with autoantibodies, including but not limited to diabetes. In further embodiments, the aptamer may prevent peripheral disease.

The present invention may further feature a method of treating a demyelinating disease in a subject in need thereof, the method comprising administering an effective amount of an aptamer that neutralizes autoantibodies in the brain. In other embodiments, the present invention features a method of treating multiple sclerosis (MS) in a subject in need thereof, the method comprising administering an effective amount of an aptamer that neutralizes myelin oligodendrocyte glycoprotein (MOG) 7 recognizing autoantibodies. In further embodiments, the present invention features a method of treating chronic inflammatory demyelinating polyradiculoneuropathy (CIDP) in a subject in need thereof, the method comprising administering an effective amount of an aptamer that neutralizes Neurofascin (NFASC) recognizing autoantibodies.

The present invention also features a neutralizing aptamer for use in a method for treatments of multiple sclerosis, wherein the aptamer neutralizes myelin oligodendrocyte glycoprotein (MOG) 7 recognizing autoantibodies. The present invention features a neutralizing aptamer for use in a method for treatment of chronic inflammatory demyelinating polyradiculoneuropathy (CIDP), wherein the aptamer neutralizes Neurofascin (NFASC) recognizing autoantibodies.

The present invention may also feature a method for identifying an aptamer for neutralizing autoantibodies. In some embodiments, the method comprises obtaining a large library of random single-stranded (DNA) sequences. In some embodiments, the method comprises incubating the ssDNA sequences with an autoantibody from the brain and eluting bound ssDNA sequences. In other embodiments, the method comprises selecting ssDNA sequences that bind to the antigen-binding region of the autoantibody. In some embodiments, the method comprises amplifying the ssDNA sequence bound to the antigen binding region of the autoantibody.

The method may further comprise removing non-specific binding of the ssDNA sequence to the Fc region of the autoantibody. In some embodiments, to remove the non-specific binding of the aptamer (ssDNA sequence), the Fc region was coupled to beads, and non-specific binders were removed by binding to the Fc region. The eluate (e.g., DNA that is not bound to the Fc region) was collected, and this was the pool of the DNA libraries that is moved forward in the selection process.

The present invention further features a method for identifying an aptamer for neutralizing autoantibodies in the brain. The method comprises obtaining a large library of random single-stranded (DNA) sequences. In some embodiments, the method comprises incubating the ssDNA sequences with an autoantibody from the brain and eluting bound ssDNA sequences. In other embodiments, the method comprises removing non-specific binding of the ssDNA sequence to the Fc region of the autoantibody and selecting ssDNA sequences that bind to the antigen binding region of the autoantibody. In some embodiments, the method comprises amplifying the ssDNA sequence bound to the antigen binding region of the autoantibody.

### Example

The following is a non-limiting example of the present invention. It is to be understood that said example is not intended to limit the present invention in any way. Equivalents or substitutes are within the scope of the present invention.

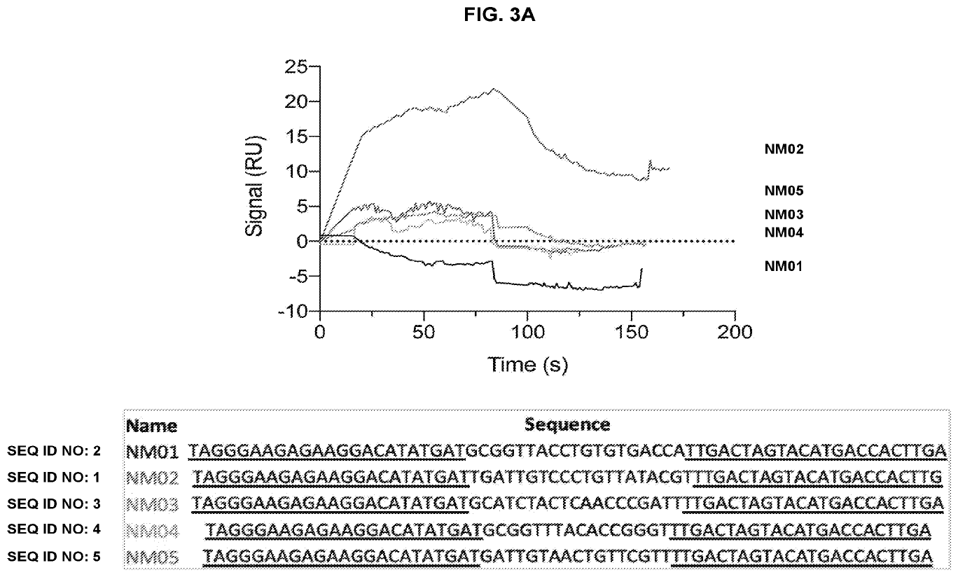

The selected ssDNA pool from different selection rounds were analyzed for improved binding towards MOG antibody using flow cytometric (FIGS. 1A, 1B and 1C) or nitrocellulose filter binding assay (FIG. 2) after SELEX optimization of aptamers. Performing flow cytometric assay on the selected DNA pool from round 6 showed 9× enrichment compared to the starting library (FIG. 1C).

Analysis of the flow data is dependent on the number of beads being injected to the instrument in each round. The best way to verify the same number of beads being used in each sample is by measuring the exact volume of the used beads, assuming homogeneous dispersion of the beads in the stock solution. Thus, a nitrocellulose filter binding assay was included as an additional method to analyze ssDNA pool enrichment. Assays were performed after 16 rounds of selection (FIG. 2) and indicated 2× enrichment of the aptamers in round 16 compared to starting a library in the presence of 10 μM of MOG antibody. Further selection rounds yielded no improvement of aptamer enrichment; therefore, aptamers from round sixteen were cloned and sequenced.

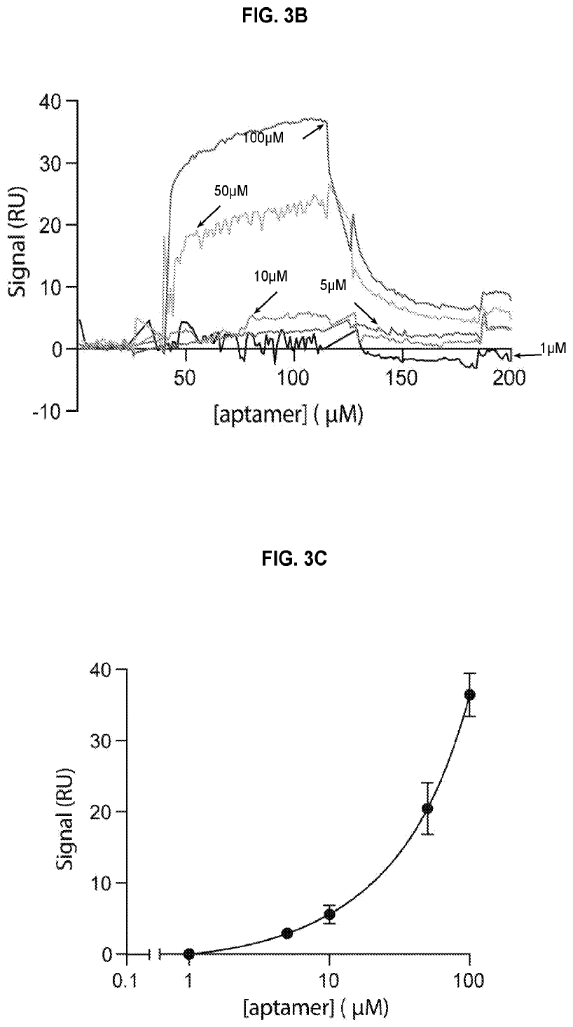

Among sequenced aptamers, sequences with the highest repetition in the enrichment pool were chosen for further studies (NM01-NM05). The binding of the chosen aptamers to the MOG antibody was measured using surface plasmon resonance (SPR) (FIG. 3A), and aptamers showing the highest binding levels at 10 μM, NM02, was chosen for affinity measurements. Binding at different aptamer concentrations (1-100 μM) to MOG antibody was measured (FIG. 3B), and the concentration-dependent curve was obtained at 110 seconds after injection, showing Kd=11±24 μM (FIG. 3C).

### Materials and Methods

Materials: All reagents were purchased from Sigma (St. Louis, MO, USA) and Thermo Fisher Scientific (Hampton, NH, USA) unless otherwise indicated. Recombinant Anti-Rat MOG Antibody (8-18C5) was purchased from Creative Biolabs (Shirley, NY, USA). The mouse IgG, Fc fragment was purchased from Jackson ImmunoResearch Laboratories Inc. (West Grove, PA, USA). The 85-bp oligonucleotide single-stranded DNA (ssDNA) library, consisting of a 40-bp randomized region flanked on either side by a 12-bp and a 13-bp primer hybridization site, was purchased from TriLink Biotechnologies (San Diego, CA, USA) to generate aptamers against MOG antibody. This library contained more than 1015 unique ssDNAs.

Systematic evolution of ligands by exponential enrichment (SELEX): The initial round of selection was done using 1 nmol of random ssDNA library because of the ability of ssDNA to form a wider variety of 3-dimensional structures compared to double-stranded DNA (dsDNA). The DNA library was heated to 90° C. for 1 min in PBS containing 1 mM MgCl2, the same concentration present in plasma, placed on ice for 15 min, and then incubated for 8 min at room temperature to allow folding. Magnetic Dynabeads™ Protein G was incubated with 200 pmol recombinant anti-rat MOG antibody for 10 minutes at room temperature as instructed by the manufacturer to immobilize the antibody. The folded DNA library was incubated with immobilized antibodies on the beads for 1 hour at 37° C. The beads were washed twice with 100 μL of PBS containing 1 mM MgCl2 using a magnetic separation rack. To the pellet was added 50 μL water, followed by 10 minutes of incubation at 90° C. to elute the bound aptamers from antibodies.

A portion of the recovered DNA was amplified by PCR (100 μL reactions) employing Taq DNA polymerase, primers at 1 μM final concentration, and incubation for 5 min at 95° C., followed by cycles of 30 s at 95° C., 30 s at 50° C., and 30 s at 72° C. for 12 cycles. The primers were designed with 5′ TAGGGAAGAGAAGGACATATGAT 3′ forward primer (SEQ ID NO: 6) and 5′ A20 -[HEG]-TCA AGT GGT CAT GTA CTA GTC AA 3′ reverse primer (SEQ ID NO: 7) (HEG indicates hexaethylene glycol spacer). The A20 -[HEG] tag, where A represents Adenine, generated a gap in size between two DNA strands after each amplification by acting as a blocker to stop polymerase activity at the 3′ terminus. The size difference between two DNA strands enabled us to separate the strands of amplified DNA by precipitation of PCR products from 10% denaturing polyacrylamide gel electrophoresis. The DNA band was cut from the gel, diced, and eluted in 300 mM sodium acetate at room temperature overnight, followed by precipitation from ethanol and quantitation by UV spectrometry.

After the first round, two steps of background subtractions were performed. This was done first using Dynabeads™ Protein G only and then second, by IgG Fc fragments immobilized on Dynabeads™ Protein G. The DNA was incubated for 30 minutes at 37° C. for each step before selection against the MOG antibody. The stringency of selection was gradually increased by lowering the concentration of the library, the concentration of MOG antibody, and the ratio of the concentration of library over the concentration of MOG antibody.

After 16 selection cycles, PCR was performed, and the resulting duplex DNA was ligated into the pUC19 cloning vector using Takara In-Fusion HD Cloning Kit, cloned, and sent to Eton Bioscience Inc. for sequencing.

Flow Cytometry: ssDNA library from each round of selection was PCR amplified using an Alexa 647 labeled forward primer with the sequence as described above. A total of 200 μL of 100 nM ssDNA selected pool was added to 1 μM of MOG antibody immobilized on 40 μL of antibody capture bead (Beckman Coulter B22804) in PBS buffer containing 1 mM MgCl2. The same volume of the beads was used for background signal subtraction (FIG. 1A), and the same concentration of the starting library was used as a control pool (FIG. 1B). The mixtures were incubated for 30 minutes at 37° C. The aptamer-MOG antibody interaction was measured as a percentage of beads showing signal in high Alexa-647 intensities using MACSQuant analyzer 10 flow cytometer (Miltenyi Biotech, Germany) (FIG. 1C). Due to the very small size of the unbound aptamers, they would not show up as signals in the performed flow cytometric assay enabling us to distinguish bound vs. unbound aptamers. The forward scatter (FSC) used for the discrimination of beads by singularity versus Alexa-647 intensity data was then analyzed by Flowlogic™ software

Radioactive filter binding assay: The ssDNA selected pool from each round of selection was PCR amplified using 32P-radioactive labeled primers. The 32P-radioactive labeled DNA was then incubated with MOG antibody at different concentrations for one hour at room temperature and passed through a nitrocellulose filter. The antibody, as well as the ssDNAs capable of binding to the antibody, were retained on the filter, and the unbound ssDNA passed through. After washing and drying, the membranes were exposed to phosphor-imaging screens for quantitation.

Surface Plasmon Resonance (SPR): Surface plasmon resonance was performed with a Biacore 3000 instrument (GE Healthcare). MOG antibody was covalently immobilized on a CM5 chip using standard amine coupling according to the manufacturer's protocol. Once the protein was immobilized (500 RU), 1 to 100 μM of aptamer NM02 was applied with a flow rate of 20 μL/min followed by 60 seconds of complex dissociation in the HBS-EP buffer. The IgG Fc fragment was immobilized on the control flow cell of the CM5 chip. The measured signals were analyzed by Biacore's Data Analysis Software and used to obtain a concentration-dependent curve and the dissociation constant of the aptamer.

### Embodiments

The following embodiments are intended to be illustrative only and not to be limiting in any way.

Embodiment 1: An aptamer that neutralizes autoantibodies in the peripheral nervous system (PNS) and/or the central nervous system (CNS).

Embodiment 2: An aptamer that neutralizes autoantibodies in the brain.

Embodiment 3: The aptamer of embodiment 1 or embodiment 2, wherein the aptamer comprises nucleotides.

Embodiment 4: The aptamer of any one of embodiments 1-3, wherein the aptamer comprises single-stranded DNA (ssDNA).

Embodiment 5: The aptamer of any one of embodiments 1-4, wherein the autoantibodies are myelin oligodendrocyte glycoprotein (MOG) 7 recognizing autoantibodies.

Embodiment 6: The aptamer of any one of embodiments 1-5, wherein the aptamer comprises SEQ ID NO: 1.

Embodiment 7: The aptamer of any one of embodiments 1-6, wherein the aptamer is at least 85% identical to SEQ ID NO: 1.

Embodiment 8: The aptamer of any one of embodiments 1-7, wherein the aptamer is at least 90% identical to SEQ ID NO: 1.

Embodiment 9: The aptamer of any one of embodiments 1-8, wherein the aptamer is at least 95% identical to SEQ ID NO: 1.

Embodiment 10: The aptamer of any one of embodiments 1-4, wherein the autoantibodies are Neurofascin (NFASC) recognizing autoantibodies.

Embodiment 11: The aptamer of any one of embodiments 1-10, further comprising peptide moiety coupled to said aptamer.

Embodiment 12: The aptamer of embodiment 11, wherein the peptide moiety increases the ability of the aptamer to cross the blood brain barrier (BBB).

Embodiment 13: The aptamer of any one of embodiments 1-12, wherein the aptamers prevent demyelination.

Embodiment 14: The aptamer of any one of embodiments 1-13, wherein the aptamer treats demyelinating diseases.

Embodiment 15: The aptamer of embodiment 14, wherein the demyelinating disease is multiple sclerosis (MS).

Embodiment 16: The aptamer of embodiment 14, wherein the demyelinating disease is chronic inflammatory demyelinating polyradiculoneuropathy (CIDP).

Embodiment 17: An aptamer that neutralizes myelin oligodendrocyte glycoprotein (MOG) 7 recognizing autoantibodies.

Embodiment 18: The aptamer of embodiment 17, wherein the aptamer comprises nucleotides.

Embodiment 19: The aptamer of embodiments 17 or 18, wherein the aptamer comprises single-stranded DNA (ssDNA).

Embodiment 20: The aptamer of any one of embodiments 17-19, wherein the aptamer comprises SEQ ID NO: 1.

Embodiment 21: The aptamer of any one of embodiments 17-20, wherein the aptamer is at least 85% identical to SEQ ID NO: 1.

Embodiment 22: The aptamer of any one of embodiments 17-21, wherein the aptamer is at least 90% identical to SEQ ID NO: 1.

Embodiment 23: The aptamer of any one of embodiments 17-22, wherein the aptamer is at least 95% identical to SEQ ID NO: 1.

Embodiment 24: The aptamer of any one of embodiments 17-23, further comprising a peptide moiety coupled to said aptamer.

Embodiment 25: The aptamer of embodiment 24, wherein the peptide moiety increases the ability of the aptamer to cross the blood brain barrier (BBB).

Embodiment 26: The aptamer of any one of embodiments 17-25, wherein the aptamers prevent demyelination.

Embodiment 27: The aptamer of any one of embodiments 17-26, wherein the aptamer treats demyelinating diseases.

Embodiment 28: The aptamer of embodiment 27, wherein the demyelinating disease is multiple sclerosis (MS).

Embodiment 29: A method of treating a demyelinating disease in a subject in need thereof, the method comprising administering an effective amount of an aptamer that neutralizes autoantibodies in the brain.

Embodiment 30: The method of embodiment 29, wherein the aptamer comprises nucleotides.

Embodiment 31: The method of embodiment 29 or embodiment 30, wherein the aptamer comprises single-stranded DNA (ssDNA).

Embodiment 32: The method of any one of embodiments 29-31, wherein the aptamer further comprises a peptide moiety coupled to said aptamer.

Embodiment 33: The method of embodiment 32, wherein the peptide moiety increases the ability of the aptamer to cross the blood brain barrier (BBB).

Embodiment 34: The method of any one of embodiments 29-33, wherein the autoantibodies are myelin oligodendrocyte glycoprotein (MOG) 7 recognizing autoantibodies or Neurofascin (NFASC) recognizing autoantibodies.

Embodiment 35: The method of any one of embodiments 29-34, wherein the demyelinating disease is multiple sclerosis (MS) or chronic inflammatory demyelinating polyradiculoneuropathy (CIDP).

Embodiment 36: A method of treating multiple sclerosis (MS) in a subject in need thereof, the method comprising administering an effective amount of an aptamer that neutralizes myelin oligodendrocyte glycoprotein (MOG) 7 recognizing autoantibodies.

Embodiment 37: A method of treating chronic inflammatory demyelinating polyradiculoneuropathy (CIDP) in a subject in need thereof, the method comprising administering an effective amount of an aptamer that neutralizes Neurofascin (NFASC) recognizing autoantibodies.

Embodiment 38: The method of embodiment 36 or embodiment 37, wherein the aptamer comprises nucleotides.

Embodiment 39: The method of any one of embodiments 36-38, wherein the aptamer comprises single-stranded DNA (ssDNA).

Embodiment 40: The method of any one of embodiments 36-39, wherein the aptamer further comprises a peptide moiety coupled to said aptamer.

Embodiment 41: The method of embodiment 40, wherein the peptide moiety increases the ability of the aptamer to cross the blood brain barrier (BBB).

Embodiment 42: A neutralizing aptamer for use in treating multiple sclerosis, wherein the aptamer neutralizes myelin oligodendrocyte glycoprotein (MOG) 7 recognizing autoantibodies.

Embodiment 43: A neutralizing aptamer for use in treating chronic inflammatory demyelinating polyradiculoneuropathy (CIDP), wherein the aptamer neutralizes Neurofascin (NFASC) recognizing autoantibodies.

Embodiment 44: The aptamer of embodiment 42 or 43, wherein the aptamer comprises nucleotides.

Embodiment 45: The aptamer of any one of embodiments 42-44, wherein the aptamer comprises single-stranded DNA (ssDNA).

Embodiment 46: The aptamer of any one of embodiments 42-45, wherein the aptamer further comprises a peptide.

Embodiment 47: The aptamer of embodiment 46, wherein the peptide increases the ability of the aptamer to cross the blood brain barrier (BBB).

Embodiment 48: A method for identifying an aptamer for neutralizing autoantibodies in a peripheral nervous system and/or central nervous system, the method comprising: (a) obtaining a large library of random ssDNA sequences; (b) incubating the ssDNA sequences with an autoantibody from the peripheral nervous system and/or central nervous system; (c) eluting bound ssDNA sequences; (d) removing non-specific binding of the ssDNA sequence to the Fc region of the autoantibody and selecting ssDNA sequences that bind to the antigen binding region of the autoantibody; (e) amplifying the bound ssDNA sequences.

Embodiment 49: A method for identifying an aptamer for neutralizing autoantibodies in a peripheral nervous system and/or central nervous system, the method comprising: (a) obtaining a large library of random ssDNA sequences; (b) incubating the ssDNA sequences with an autoantibody from the peripheral nervous system and/or central nervous system; (c) eluting bound ssDNA sequences; (d) selecting ssDNA sequences that bind to the antigen binding region of the autoantibody; (e) amplifying the bound ssDNA sequences.

Embodiment 50: The method of 49, further comprising removing non-specific binding of the ssDNA sequence to the Fc region of the autoantibody.

Embodiment 51: The method of embodiment 49 or embodiment 50, wherein the autoantibody is from a brain.

Embodiment 52: A method for identifying a high-affinity aptamer against myelin oligodendrocyte glycoprotein (MOG) 7 recognizing autoantibodies, the method comprising: (a) obtaining a large library of random ssDNA sequences; (b) incubating the ssDNA sequences with MOG 7 recognizing autoantibodies; (c) eluting bound ssDNA sequences; (d) removing non-specific binding of the ssDNA sequence to the Fc region of the MOG 7 recognizing autoantibodies and selecting ssDNA sequences the bind to the antigen binding region of the MOG 7 recognizing autoantibodies; (e) amplifying the bound ssDNA sequences.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. An aptamer that neutralizes autoantibodies in the peripheral nervous system (PNS) and/or the central nervous system (CNS).

2. The aptamer of claim 1, wherein the aptamer comprises nucleotides.

3. The aptamer of claim 1, wherein the aptamer comprises single-stranded DNA (ssDNA).

4. The aptamer of claim 1, wherein the autoantibodies are myelin oligodendrocyte glycoprotein (MOG) 7 recognizing autoantibodies or Neurofascin (NFASC) recognizing autoantibodies.

5. The aptamer of claim 1, wherein the aptamer comprises SEQ ID NO: 1.

6. The aptamer of claim 1, wherein the aptamer is at least 85%, at least 90%, or at least 95% identical to SEQ ID NO: 1.

7.-9. (canceled)

10. The aptamer of claim 1, further comprising peptide moiety coupled to said aptamer, wherein the peptide moiety increases the ability of the aptamer to cross the blood brain barrier (BBB).

11. (canceled)

12. The aptamer of claim 1, wherein the aptamers prevent demyelination.

13. The aptamer of claim 1, wherein the aptamer treats demyelinating diseases.

14. The aptamer of claim 13, wherein the demyelinating disease is multiple sclerosis (MS) or chronic inflammatory demyelinating polyradiculoneuropathy (CIDP).

15.-27. (canceled)

28. A method of treating a demyelinating disease in a subject in need thereof, the method comprising administering an effective amount of an aptamer that neutralizes autoantibodies in the brain.

29. The method of claim 28, wherein the aptamer comprises nucleotides.

30. The method of claim 28, wherein the aptamer comprises single-stranded DNA (ssDNA).

31. The method of claim 28, wherein the aptamer further comprises a peptide moiety coupled to said aptamer, wherein the peptide moiety increases the ability of the aptamer to cross the blood brain barrier (BBB).

32. (canceled)

33. The method of claim 28, wherein the autoantibodies are myelin oligodendrocyte glycoprotein (MOG) 7 recognizing autoantibodies or Neurofascin (NFASC) recognizing autoantibodies.

34. The method of claim 28, wherein the demyelinating disease is multiple sclerosis (MS) or chronic inflammatory demyelinating polyradiculoneuropathy (CIDP).

35.-47. (canceled)

48. A method for identifying an aptamer for neutralizing autoantibodies in a peripheral nervous system and/or central nervous system, the method comprising:
a) obtaining a large library of random single-stranded DNA (ssDNA) sequences;
b) incubating the ssDNA sequences with an autoantibody from the peripheral nervous system and/or central nervous system;
c) eluting bound ssDNA sequences;
d) selecting ssDNA sequences that bind to the antigen binding region of the autoantibody;
e) amplifying the bound ssDNA sequences.

49. The method of 48, further comprising removing non-specific binding of the ssDNA sequence to the Fc region of the autoantibody prior to amplifying the bound ssDNA sequences.

50. The method of claim 48, wherein the autoantibody is from a brain.

51. (canceled)

52. The method of claim 48, wherein the the autoantibodies are myelin oligodendrocyte glycoprotein (MOG) 7 recognizing autoantibodies or Neurofascin (NFASC) recognizing autoantibodies
